(19) United States
(12) Patent Application Publication
(10) Pub. No.: US 2016/0168473 A1
(43) Pub. Date: Jun. 16, 2016

(54) METHODS FOR PRODUCING ALKANES FROM BIOMASS

(71) Applicant: EAST CHINA UNIVERSITY OF SCIENCE AND TECHNOLOGY, Shanghai (CN)

(72) Inventors: Yanqin WANG, Shanghai (CN); Qineng XIA, Shanghai (CN); Yu ZHANG, Shanghai (CN); Guanzhong LU, Shanghai (CN); Jiawen REN, Shanghai (CN); Xiaohui LIU, Shanghai (CN); Xueqing GONG, Shanghai (CN); Yun GUO, Shanghai (CN); Yanglong GUO, Shanghai (CN); Junsong WANG, Shanghai (CN); Zhigang ZHANG, Shanghai (CN); Wangcheng ZHAN, Shanghai (CN)

(73) Assignee: EAST CHINA UNIVERSITY OF SCIENCE AND TECHNOLOGY, Shanghai (CN)

(21) Appl. No.: 14/907,327

(22) PCT Filed: Jul. 24, 2013
(86) PCT No.: PCT/CN2013/079958

§ 371 Date: Jan. 25, 2016

(51) Int. Cl. C10G 1/06; B01J 23/652; C07C 29/00; B01J 29/48; C10G 1/02; C07D 313/04; B01J 27/053; B01J 23/89

(52) U.S. Cl. CPC C10G 1/06; B01J 27/053; B01J 23/6527; B01J 23/8926; B01J 29/48; C10G 1/02; C07D 313/04; C07C 29/00

(57) ABSTRACT

Methods and systems for producing one or more alkanes from biomass by hydrodeoxygenation are disclosed. Biomass may be converted to one or more alkanes by heating the biomass with a catalyst mixture that includes a noble metal and at least one of a transition metal and derivative thereof. The catalyst mixture may further include a solid acid. Heating may be performed at a single temperature and pressure.

### BACKGROUND

The rapid depletion of fossil fuels has been a driving force to identify alternative sources for the production of alkanes. Biomass provides one such source for the production of alkanes. Biomass is carbon, hydrogen and oxygen based, and encompasses a wide variety of materials including plants, wood, garbage, paper, crops and waste products. Other biomass sources may include waste materials, such as forest residues, municipal solid wastes, waste paper, and crop residues.

The main components of biomass are cellulose, starch and hemi-cellulose, with cellulose making up about 36-42% of the dry weight of non-food biomass feedstock, and hemicellulose making up about 21-25% of the dry weight of non-food biomass feedstock. Cellulose consists of a linear chain of several hundred to over ten thousand β(1→4) linked D-glucose units. Cellulose is the structural component of the primary cell walls of green plants and many forms of algae, making it one of the most common organic compounds on Earth. Starch is similar to cellulose and contains a large number of glucose units joined by α(1→4) linkages. Starch is produced by all green plants as an energy source and thus is the most common carbohydrate in the human diet. Hemicellulose is formed from both 6-carbon (C6) sugars and 5-carbon (C5) sugars. Hemicellulose monomers may include one or more of glucuronic acid, galactose, mannose, rhanmose, arabinose, most of the D-pentose sugars and some L-sugars, with xylose being present in the largest amount.

The safe disposal of organic waste or biomass, which can be contaminating to the environment, has been recognized as a significant health and economic issue for many years. The ability to dump waste materials into the oceans or landfills is no longer a favored mechanism for disposal. Not only do landfills face a limitation on space and require significant energy to transport and deposit materials, but they are recognized as potential health hazards and can be ecologically destructive at their locations and adjacent land areas, in part due to underground seepage of these materials. Therefore, methods to reduce biomass disposal by converting them to economical products is important. Thus, there remains a need to develop methods and processes that use biomass or organic waste as a source for producing alkanes,

### SUMMARY

The present disclosure provides methods to produce alkanes from biomass by hydrodeoxygenation. In one embodiment, a method of converting biomass to one or more alkanes may involve heating the biomass and a catalyst mixture to form the one or more alkanes, wherein the catalyst mixture includes a noble metal and at least one of a transition metal and a transition metal compound, and wherein the heating is performed at a single temperature and pressure.

In an additional embodiment, a catalyst mixture may include a noble metal, at least one of a transition metal and a transition metal compound, and a solid acid. In some embodiments, the catalyst mixture may be configured to convert biomass to one or more alkanes,

In a further embodiment, a reactor system may include a reactor vessel configured to receive a biomass and a catalyst mixture, wherein the catalyst mixture comprises a noble metal and at least one of a transition metal and a transition metal compound; and a heater configured to heat the biomass and the catalyst mixture in the reactor vessel to produce the one or more alkanes.

### BRIEF DESCRIPTION OF THE FIGURES

FIG. 1 shows product components obtained from direct hydrodeoxygenation of corncob at 200° C., 3.0 MPa, using 5% Pt-20% W/Al₂O₃ as catalyst and water as solvent, according to an embodiment. The x-axis indicates the various product components, and the y-axis indicates percent yield.

### DETAILED DESCRIPTION

This disclosure is not limited to the particular systems, devices and methods described, as these may vary. The terminology used in the description is for the purpose of describing the particular versions or embodiments only, and is not intended to limit the scope.

As used herein, “solid acid” refers to a Lewis acid or a Brönsted acid which includes oxides, hydroxides, halides, sulfates, phosphates or composites of a metal to catalyze a dehydration step.

As used herein, “biomass” refers to any organic material produced by plants such as leaves, roots, seeds and stalks), and microbial and animal metabolic wastes.

The present disclosure provides methods to produce one or more alkanes from biomass. The one or more alkanes may include liquid alkanes. In some embodiments, a method of converting biomass to one or more alkanes may include heating the biomass and a catalyst mixture to form the one or more alkanes, wherein the catalyst mixture includes a noble metal and at least one of a transition metal and a transition metal compound, and wherein the heating is performed at a single temperature and pressure. In some embodiments, the biomass or biomass-derived materials include, but are not limited to, a carbohydrate, polysaccharide, monosaccharide, disaccharide, cellulose, lignin, starch, pentose, and any combinations thereof. In some embodiments, the biomass may include, but are not limited to, organic waste, food processing by-product, vegetable mixtures, fruit mixtures, corncob, rice straw, rice husk, tapioca, sawdust, pone wood, bagasse, corn stover, sugar cane, hemicellulose, glycogen, lactose, sucrose, maltose, cellobiose, hexose, maize straw, wheat bran, rice hulls, grains, plant matter, animal product, beef suet, aldol adducts of furfural, 5-hydromethylfurfural (HMF) with acetone, and any combinations thereof. The amount of biomass used as the starting material may vary depending on the scale of the commercial process, or size of the mixing reaction vessel. Exemplary alkanes that may be obtained by the methods described in the disclosed embodiments include, but are not: limited to, linear or branched C₁ to C₁₅ alkane, C₁ to C₁₅ alkyl alcohol, C₁ to C₆ cycloalkane, C₁ to C₆ substituted cycloalkane, cyclic ether, and any combination thereof. Typical alkanes, alkanols, and other cyclic compounds that can be produced by the methods described in the disclosed embodiments include, but are not limited to, methane, ethane, propane, butane, pentane, hexane, alkyl cyclohexane, 1-hexanol, cyclohexyl alcohol, and any combinations thereof. Alkanes are typically liquids, but may be solids, liquids, or gases, depending on the particular temperature and pressure of their environment.

The catalyst mixture disclosed in the embodiments herein may be a mixture of one or more noble metals, and one or more transition metals and transition metal compounds. Non-limiting examples of a noble metal include Au, Pt, Pd, Ir, Os, Ag, Rh, Ru, and any combination thereof. In some embodiments, the transition metal may be an elemental transition metal. In some embodiments, the transition metal compound is a transition metal oxide, transition metal phosphate, transition metal sulfate, or any combinations thereof. The transition metal or transition metal compounds may possess redox properties and catalyze the cleavage of a C—O bond. Non-limiting examples of transition metals include Ti, Zr, Hf, V, Nb, Ta, Cr, Mn, Mo, W, Re, Co, Ni, Cu, or any combination thereof. Non limiting examples of transition metal compounds include oxides, sulfates, and phosphates of Ti, Zr, Hf, V, Nb, Ta, Cr, Mn, Mo, W, Re, Co, Ni, Cu, or any combination thereof.

In some embodiments, the catalyst mixture may further include a solid acid. For example, the solid acid may be required if the noble metal and the at least one of the transition metal and the transition metal compound in the catalyst mixture do not have acidic properties that are usually required for catalysis. The solid acid may be a Lewis acid or a Brönsted acid, which may include, for example, metal oxides, metal hydroxides, metal halides, metal sulfates, metal phosphates, or any combination thereof. In some embodiments, the solid acid may be a zeolite, an ion-exchange resin, a clay, and the like.

A suitable solid acid may be a solid material that demonstrates sufficient acidity to protonate pyridine. The use of pyridine as a probe molecule coupled with Fourier transform Infra-Red (FTIR) spectroscopy is routinely used to investigate the acidity of solids. Pyridine is protonated by reaction with Brönsted acid sites of sufficient strength. When pyridine interacts with such acid sites on a surface, an absorption at about 1546 cm⁻¹ can be measured by FTIR, allowing quantification of Brönsted acid sites. The pKa of the conjugate acid of pyridine is 5.2. As such, using any acid with a pKa less than 5.2 will result in some degree of protonation of pyridine. Suitable solid acids therefore may have a pKa<5.2 and may be active for the hydrodeoxygenation of biomass and other carbohydrates at the selected reaction conditions. Non-limiting examples of solid acids include ZrO(SO₄), TiCl₃, Ti₂(SO₄)₃, CrPO₄, CrCl₂, MnCl₂, Mn₃(PO₄)₂, Co₃(PO₄)₂, CoSO₄, MoO₃, Mo(SO)₃, TaF₅, W(PO)₄, Al₂O₃, NbPO₄, Nb₂O₅, NbSO₄, TaCl₂, TaSO₄, Ta₃PO₄, SnPO4, SnCl2, SnSO₄, VPO₄, VSO₄, ZnSO₄, ZnCl₂, ZnPO₄, and any combination thereof.

In some embodiments, the catalyst may be a mixture of one or more noble metals, at least one of: one or more elemental transition metals and one or more transition metal compounds, and one or more solid acids. In some embodiments, the catalyst may be a mixture of one or more noble metals, one or more elemental transition metals, and one or more solid acids. In some embodiments, the catalyst may be a mixture of one or more noble metals, one or more transition metal oxides, and one or more solid acids. In some embodiments, the catalyst may be a mixture of one or more noble metals, one or more transition metal sulfates, and one or more solid acids. In some embodiments, the catalyst may be a mixture of one or more noble metals, one or more transition metal phosphates, and one or more solid acids. In some embodiments, the catalyst may be a mixture of one or more noble metals and one or more transition metal oxides. In some embodiments, the catalyst may be a mixture of one or more noble metals and one or more transition metal sulfates. In some embodiments, the catalyst may be a mixture of one or more noble metals and one or more transition metal phosphates. Non-limiting examples of catalyst mixtures include, but are not limited to, Pd/CuO/SiO₂, Rh/Ta₂O₅/Al₂O₃, Pd—Re/ZSM-5, Pt—W/Al₂O₃, Pt—Co/Mn₃(PO₄)₂, Pd/ZrOSO₄, PtiNbOPO₄, Pt/Nb₂O₅, Pd/WO₃, or any combination thereof.

In some embodiments, when the catalyst mixture is a mixture of a noble metal, a transition metal, and a solid acid, the amount of noble metal in the catalyst mixture may be in the range of about 0.1 weight percent to about 10 weight percent, about 0.1 weight percent to about 8 weight percent, about 0.1 weight percent to about 5 weight percent, about 0.1 weight percent to about 2.5 weight percent, or about 0.1 weight percent to about 1 weight percent of the catalyst mixture. Specific examples include about 0.1 weight percent, about 1 weight percent, about 2.5 weight percent, about 5 weight percent, about 7 weight percent, about 10 weight percent, and ranges between any two of these values (including their endpoints).

In some embodiments, when the catalyst mixture is a mixture of a noble metal, a transition metal, and a solid acid, the amount of transition metal in the catalyst mixture may be in the range of about 1 weight percent to about 30 weight percent, about 1 weight percent to about 20 weight percent, about 1 weight percent to about 10 weight percent, about 1 weight percent to about 5 weight percent, or about 1 weight percent to about 2 weight percent of the catalyst mixture. Specific examples include about 1 weight percent, about 5 weight percent, about 15 weight percent, about 20 weight percent, about 25 weight percent, about 30 weight percent, and ranges between any two of these values (including their endpoints). Additionally, if the solid acid is absent in the catalyst mixture, the amount of transition metal in the catalyst mixture may be higher, such as about 1 weight percent to about 99.9 weight percent, about 1 weight percent to about 90 weight percent, about 1 weight percent to about 70 weight percent, about 1 weight percent to about 50 weight percent, or about 1 weight percent to about 20 weight percent of the catalyst mixture. Specific examples include about 1 weight percent, about 25 weight percent, about 45 weight percent, about 60 weight percent, about 85 weight percent, about 99.9 weight percent, and ranges between any two of these values (including their endpoints).

In some embodiments, when the catalyst mixture is a mixture of a noble metal, a transition metal, and a solid acid, the amount of solid acid in the catalyst mixture may be in the range of about 60 weight percent to about 99 weight percent, about 60 weight percent to about 90 weight percent, about 60 weight percent to about 80 weight percent, about 60 weight percent to about 70 weight percent, or about 60 weight percent to about 65 weight percent of the catalyst mixture. Specific examples include about 60 weight percent, about 70 weight percent, about 75 weight percent, about 80 weight percent, about 95 weight percent, about 99 weight percent, and ranges between any two of these values (including their endpoints).

Exemplary catalyst mixtures include, but are not limited to, 4% Pd/96% ZrOSO₄, 4% Pt-20% W/76% Al₂O₃, 5% Pt-20% W/75% Al₂O₃, 4% Pd/20% Cu0/76% SiO₂, 5% Rh/15% Ta₂O₅/80% Al₂O₃, 5% Pd-5% Re/90% ZSM-5, 4% Pt-4% W/92?.4Al₂O₃, 5% Pt-10% Co/85% Mn₃(PO₄)₂, 85% Mn₃(PO₄)₂, 5% Pd/95% ZrOSO₄, 4% Pt/96% NbOPO₄, and the like.

The catalyst mixtures described in the embodiments herein may be unsupported or may be supported by distribution over a surface of a support in a manner that maximizes the surface area of the catalytic reaction. A suitable support may be selected from any conventional support, such as a silica-alumina cogel, silica, a transition alumina, such as gamma, delta or theta altiminas, carbon, titania, zirconia, and sulphated zirconia. Mixtures of these support m terials may also be used. The catalyst mixture may also be supported on at least a portion of the solid acid catalyst.

Supported catalyst mixtures may be formed by contacting or impregnating the support with a solution of the catalyst mixture, and drying. In some embodiments, the dried material may be calcinated. Alternative methods may include precipitation of a compound of metals in the catalyst mixture onto the support, or with the support. Alternatively, the catalyst mixture may be introduced Onto the support by ion-exchange if the selected support is facilitates such methods.

In some embodiments, a reactor system may include a reactor vessel configured to receive a biomass and a catalyst mixture as described in the disclosed embodiments, and a heater configured to heat the biomass and the catalyst mixture in the rector vessel to produce one or more alkanes. In some embodiments, the reactor vessel may contain the biomass and the catalyst mixture, wherein the catalyst mixture includes a noble metal and at least one of a transition metal and a transition metal compound; and a heater configured to heat the biomass and the catalyst mixture in the reactor vessel to produce the one or more aikanes. In some embodiments, the catalyst mixture may further contain a solid acid. The processes described herein may be performed in a batch reactor or in a continuous flow reactor. In the batch reactor, the biomass material may be placed in the reactor at the beginning of the reaction period, after which the reactor is closed for the entire period without adding additional components. In the continuous flow reactor, the reactor may be filled continuously with fresh material and also emptied continuously. The reactor vessel may be configured to receive the biomass, the catalyst mixture, or any other reactant as will be described in the paragraphs below such as hydrogen (H₂) gas, separately or in any combination.

The reactor system may be connected to a thermoelectric couple, a pressure gauge, a temperature controller, a cooling system, a mechanical stirrer, a plurality of gas valves, or any combination thereof. In some embodiments, a batch operation may be performed in a conventional autoclave. The reactants may be added to the chamber in any suitable manner or in any suitable order. In one embodiment, the catalyst mixture is added first to the biomass to form a biomass-catalyst mixture, and thereafter, fed with hydrogen gas. In some embodiments, the conversion of biomass to alkanes may be carried out in a single step. Alternatively, it may be operated in two or more steps.

In some embodiments, biomass including carbohydrates and other biomass derived materials may be mixed into any aqueous reaction medium, including water, methanol, hexane, or any combination thereof. In some embodiments, a solvent may not be required, such as when using raw biomass. Thereafter, the biomass may be contacted with either hydrogen or hydrogen mixed with a suitable gas along with the catalyst mixture under conditions sufficient to form a hydrogenated product, such as alkanes. The gas may be introduced into the reaction chamber under pressure, which may vary with factors such as the nature of the reactants and the catalyst mixture employed. The rate at which gas is introduced to the reaction vessel may also vary according to the same factors.

In some embodiments, the biomass and the catalyst mixture may be heated to an elevated temperature. Examples of elevated temperatures include about: 150° C. to about 250° C., about 150° C. to about 225° C., about 150° C. to about 200° C., or about 150 ° C. to about 175° C. Specific examples include about 150° C., about 175° C., about 200° C., about 225° C., about 250° C., and ranges between any two of these values (including their endpoints). The heating step can generally be performed for any suitable period of time. Suitable time periods for this reaction process may include from about 2 hours to about 48 hours, about 2 hours to about 36 hours, about 2 hours to about 24 hours, about 2 hours to about 12 hours, about 2 hours to about 6 hours, or about 2 hours to about 4 hours. Specific examples include about 2 hours, about 4 hours, about 6 hours, about 12 hours, about 24 hours, about 30 hours, about 48 hours, and ranges between any two of these values (including their endpoints). In some cases, longer periods of times may be used.

In some embodiments, the biomass and the catalyst mixture may be heated in the presence of hydrogen (H₂) under a pressure of about 1 MPa to about 20 MPa, about 1 MPa to about 15 MPa, about 1 MPa to about 10 Pv1Pa, or about 1 MPa to about 5 MPa. Specific examples include about 1 MPa, about 2.5 MPa, about 5 MPa, about 10 MPa, about 15 MPa, about 20 MPa, and ranges between any two of these values (including their endpoints). It is, however, understood that higher and lower temperatures and pressures than those described above may be used when deemed necessary or desirable to optimize results.

Alkanes that are generated as described herein may optionally be purified by any method known in the art. For example, alkanes may be purified by using one or more methods including solvent extraction, distillation, and the like. Solvents such as aqueous trimethylamine, water, or aqueous NaOH may be used for the extraction. Alkanes may be further purified by electrodialysis, reverse-osmosis, supercritical CO₂ extraction or any other methods known in the art.

### EXAMPLES

### Example 1

### One-Step Production of Alkanes from Cellulose

A 4 wt % Pd 96 wt % ZrOSO₄ catalyst mixture was used for the hydrodeoxygenation of cellulose, which was prepared by impregnation of ZrOSO₄ with an aqueous solution of Pd(NO₃)₂.H₂O. After the impregnation, the catalyst mixture was dried at 100° C. for 12 hours, followed by calcination in air at 500° C. for 3 hours. Reactions 1 to 5 as shown in Table 1 were carried out in batch reactors for 16 hours in the presence of hydrogen (H₂), and at different temperatures and pressures, with methanol as a solvent. About 0.5 grams of cellulose was used in this experiment. The contents of the reaction mixture were analyzed by gas chromatograph (GC) and mass spectrometry, and quantified by GC equipped with FID detector using dodecane as internal standard substance. The product distribution of alkalines derived from the cellulose conversion under the different reaction conditions are summarized in Table 1 below. It can be seen from Table 1 that reactions performed at 190-200° C. and 5 MPa (Reactions #2 and #3) provided better yields of hexane.

TABLE 1

| | | | Conver- | Hexane | Pentane | Butane | Propane | Ethane | Methane | Total alkanes |
|---|---|---|---|---|---|---|---|---|---|---|
| | Temp. | Pressure | ion | (weight | (weight | (weight | (weight | (weight | (weight | (weight |
| Reaction | (° C.) | (MPa) | (%) | %) | %) | %) | %) | %) | %) | %) |
| 1 | 170 | 5 | 86 | 11.8 | 2.0 | 0.5 | 0.2 | 0.1 | 0.8 | 15.4 |
| 2 | 190 | 5 | >99 | 71.6 | 7.8 | 1.3 | 0.5 | 0.5 | 3.1 | 84.8 |
| 3 | 200 | 5 | >99 | 69.7 | 8.7 | 1.5 | 0.5 | 0.4 | 3.5 | 82.3 |
| 4 | 200 | 2 | 98 | 47.1 | 11.6 | 2.5 | 0.6 | 0.5 | 4.6 | 66.9 |
| 5 | 200 | 3 | >99 | 54.5 | 8.9 | 1.4 | 0.3 | 0.3 | 3.7 | 69.1 |

### Example 2

### Biomass Conversion to Alkanes

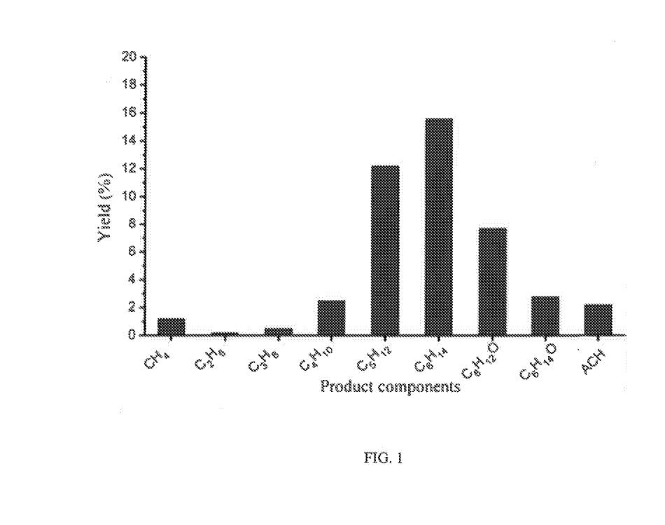

A 5 wt % Pt-20 wt % W/75 wt % Al₂O₃ was used as a catalyst mixture to convert biotnass (corncob) to alkanes. The reaction was carried out in a 50-mL stainless steel autoclave (200° C., 3.0 MPa) for 24 hours, with water as a solvent. About 0.5 gram of pre-treated corncob was used in this process. The corncob powder was obtained by sawing the dried corncob, and the corncob sawdust was ball-milled (800 r/min) for 6 hours and dried at 100° C. for 24 h before use. The resulting alkanes and their respective yields are shown in FIG. 1. In addition to C1-C6 alkanes, C6 cyclic ether (C₆H₁₂O), hexyl alcohol (C₆H₁₄O), and alkylcyclohexarte (ACH, from hydrodeoxygenation of lignin) were also obtained.

### Example 3

### Production of Alkanes from Food Waste

A 4 wt % Pd/20 wt % CuO/76 wt % SiO₂ catalyst mixture is used for the hydrodeoxygcnatio:n. of food waste (mixture of carrots, onions, potatoes and beef suet in equal proportions by weight). The reaction is carried out in a batch reactor for 16 hours in the presence of hydrogen (H₂), at a temperature of 200° C. and at a pressure of 5 MPa, with methanol as the solvent. About 5 grams of food waste is used in this experiment. The contents of the reaction product will be analyzed by GC/MS. About 55-75 weight % of the reaction product will be made up of hexane.

### Example 4

### Production of Alkanes from Rice Hulls

A 5 wt % Pd-5 wt % Re/90 wt % ZSM-5 catalyst mixture is used for the hydrodeoxygenation of rice hulls. The reaction is carried out in a batch reactor for 16 hours in the presence of hydrogen (H₂), at a temperature of 200° C. and at a pressure of 5 MPa, with methanol as the solvent. About 5 grams of rice hulls are used in this experiment. The contents of the reaction mixture will be analyzed by GC/MS. About 55-75 weight % of the reaction product will be made up of hexane.

In the above detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be used, and other changes may be made, without departing from the spirit or scope of the subject matter presented herein. It will be readily understood that the aspects of the present disclosure, as generally described herein, and illustrated in the Figures, can be arranged, substituted, combined, separated, and designed in a wide variety of different configurations, all of which are explicitly contemplated herein.

The present disclosure is not to be limited in terms of the particular embodiments described in this application, which are intended as illustrations of various aspects. Many modifications and variations can be made without departing from its spirit and scope, as will be apparent to those skilled in the art. Functionally equivalent methods and apparatuses within the scope of the disclosure, in addition to those enumerated herein, will be apparent to those skilled in the art from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds, compositions o biological systems, which can, of course, vary. It is also to be understood that the terminology used herein is for the purpose of describing particular embodiments only, and is not intended to be limiting.

As used in this document, the singular forms “a,” “an,” and “the” include plural references unless the context clearly dictates otherwise. Unless defined otherwise, all technical and scientific terms used herein have the same meanings as commonly understood by one of ordinary skill in the art. Nothing in this disclosure is to be construed as an admission that the embodiments described in this disclosure are not entitled to antedate such disclosure by virtue of prior invention. As used in this document, the term “comprising” means “including, but not limited to.”

While various compositions, methods, and devices are described in terms of “comprising” various components or steps (interpreted as meaning “including, but not limited to”), the compositions, methods, and devices can also “consist essentially of” or “consist of” the various components and steps, and such terminology should be interpreted as defining essentially closed-member groups.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

Various of the above-disclosed and other features and functions, or alternatives thereof, may be combined into many other different systems or applications. Various presently unforeseen or unanticipated alternatives, modifications, variations or improvements therein may be subsequently made by those skilled in the art, each of which is also intended to be encompassed by the disclosed embodiments.

1. A method of converting biomass to one or more alkanes, the method comprising:
heating the biomass and a catalyst mixture for forming the one or more alkanes, wherein the catalyst mixture comprises:
a noble metal;
at least one of a transition metal and a transition metal compound; and
a solid acid,
wherein the heating is performed at a temperature of about 150° C. to about 250° C. and under a pressure.

2. The method of claim 1, wherein the heating comprises heating the biomass comprising a carbohydrate, polysaccharide, monosaccharide, disaccharide, cellulose, lignin, starch, pentose, or any combination thereof.

3. The method of claim 1, wherein the heating comprises heating the biomass comprising organic waste, food processing by-product, a vegetable mixture, a fruit mixture, corncob, rice straw, rice husk, tapioca, sawdust, pone wood, bagasse, corn stover, sugar cane, hemicellulose, glycogen, lactose, sucrose, maltose, cellobiose, hexose, maize straw, wheat bran, rice hulls, grains, plant matter, animal product, beef suet, aldol adducts of furfural, or any combination thereof.

4. The method of claim 1, wherein the forming comprises forming the one or more alkanes comprising a C₁ to C₁₅ alkane, C₁ to C₁₅ alkyl alcohol, C₁ to C₆ cycloalkane, C₁ to C₆ substituted cycloalkane, cyclic ether, or any combination thereof.

5. The method of claim 1, wherein the forming comprises forming the one or more alkanes comprising methane, ethane, propane, butane, pentane, hexane, alkyl cyclohexane, 1-hexanol, cyclohexyl alcohol, or any combination thereof.

6. The method of claim 1, wherein the heating comprises heating the catalyst mixture comprising the noble metal, wherein the noble metal is Au, Pt, Pd, Ir, Os, Ag, Rh, Ru, or any combination thereof.

7. The method of claim 1, wherein the heating comprises heating the catalyst mixture comprising the transition metal compound, wherein the transition metal compound is a transition metal oxide, a transition metal phosphate, a transition metal sulfate, or any combination thereof.

8. The method of claim 1, wherein the heating comprises heating the catalyst mixture comprising the transition metal, wherein the transition meal is Ti, Zr, Hf, V, Nb, Ta, Cr, Mn, Mo, W, Re, Co, Ni, Cu, or any combination thereof.

9. (canceled)

10. The method of claim 1, wherein the heating comprises heating the catalyst mixture having the solid acid, wherein the solid acid comprises a metal oxide, a metal hydroxide, a metal halide, a metal sulfate, a metal phosphate, zeolite, an ion-exchange resin, or any combination thereof.

11. The method of claim 1, wherein the heating comprises heating the catalyst mixture having the solid acid, wherein the solid acid comprises ZrO(SO₄), TiCl₃, Ti₂(SO₄)₃, CrPO₄, CrCl₂, MnCl₂, Mn₃(PO₄)₂, Co₃(PO₄)₂, CoSO₄, MoO₃, Mo(SO)₃, TaF₅, W(PO)₄, Al₂O₃, NbPO₄, Nb₂O₅, Nb SO₄, TaCl₂, TaSO₄, Ta₃PO₄, SnPO4, SnCl2, SnSO₄, VCl₂, VPO₄, VSO₄, ZnSO₄, ZnCl₂, ZnPO₄, or any combination thereof.

12. The method of claim 1, wherein the heating comprises heating the catalyst mixture having the noble metal, wherein the noble metal is present in the catalyst mixture at a concentration of about 0.1% to about 10% by weight.

13. The method of claim 1, wherein the heating comprises heating the catalyst mixture having the transition metal, wherein the transition metal is present in the catalyst mixture at a concentration of about 1% to about 70% by weight.

14. The method of claim 1, wherein the heating comprises heating the catalyst mixture having the solid acid, wherein the solid acid is present in the catalyst mixture at a concentration of about 60% to about 99% by weight.

15. The method of claim 1, wherein the heating comprising heating the catalyst mixture, wherein the catalyst mixture is Pd/CuO/SiO₂, Rh/Ta₂O₅/Al₂O₃, Pd—Re/ZSM-5, Pt—W/Al₂O₃, Pt—Co/Mn₃(PO₄)₂, Pd/ZrOSO₄, Pt/NbOPO₄ Pt/Nb₂O₅ Pd/WO₃, or any combination thereof.

16. (canceled)

17. The method of claim 1, wherein the heating comprises heating the catalyst mixture, wherein the catalyst mixture is Pd/ZrOSO₄, and the Pd is present in the catalyst mixture at a concentration of about 4% by weight.

18. The method of claim 9, wherein the heating comprises heating the catalyst mixture, wherein the catalyst mixture is Pt—W/Al₂O₃, and the Pt is present in the catalyst mixture at a concentration of about 5% by weight of the catalyst mixture, and the W is present in the catalyst mixture at a concentration of about 20% by weight of the catalyst mixture.

19. (canceled)

20. The method of claim 1, wherein the heating comprises heating for about 2 hours to about 48 hours.

21. The method of claim 1, further comprising heating the biomass and the catalyst mixture in the presence of hydrogen (H₂) under a pressure of about 1 MPa to about 20 MPa.

22. The method of claim 1, wherein the heating comprises heating the biomass and the catalyst mixture in the presence of hydrogen (H₂) under a pressure of about 5 MPa and at a temperature of about 190° C. for about 16 hours.

23. The method of claim 1, further comprising heating the biomass and the catalyst mixture in the presence of at least one solvent.

24. The method of claim 1, wherein the heating further comprises heating in the presence of water, methanol, hexane, or any combination thereof.

25. The method of claim 1, wherein the heating is performed in a batch reactor or a continuous flow reactor.

26. The method of claim 1, wherein the heating is performed in a single-step process.

27. A catalyst mixture comprising:
a noble metal;
at least one of a transition metal and a transition metal compound; and
a solid acid, wherein the solid acid is present in the catalyst mixture at a concentration of about 20% to about 90% by weight.

28.-30. (canceled)

31. The catalyst mixture of claim 27, wherein the noble metal is Au, Pt, Pd, Ir, Os, Ag, Rh, Ru, or any combination thereof.

32. The catalytic mixture of claim 27, wherein the transition metal compound is a transition metal oxide, a transition metal phosphate, a transition metal sulfate, or a combination thereof.

33. The catalyst mixture of claim 27, wherein the solid acid comprises a metal oxide, a metal hydroxide, a metal halide, a metal sulfate, a metal phosphate, zeolite, an ion-exchange resin, or any combination thereof.

34. The catalyst mixture of claim 27, wherein the solid acid comprises ZrO(SO₄), TiCl₃, Ti₂(SO₄)₃, CrPO₄, CrCl₂, MnCl₂, Mn₃(PO₄)₂, Co₃(PO₄)₂, CoSO₄, MoO₃, Mo(SO)₃, TaF₅, W(PO)₄, Al₂O₃, NbPO₄, Nb₂O₅, Nb SO₄, TaCl₂, TaSO₄, Ta₃PO₄, SnPO4, SnC12, SnSO₄, VCl₂, VPO₄, VSO₄, ZnSO₄, ZnCl₂, ZnPO₄, or any combination thereof.

35. The catalyst mixture of claim 27, wherein the noble metal is present in the catalyst mixture at a concentration of about 0.1% to about 10% by weight.

36. The catalyst mixture of claim 27, wherein the transition metal is present in the catalyst mixture at a concentration of about 1% to about 30% by weight.

37. (canceled)

38. The catalyst mixture of claim 27, wherein the catalyst mixture is Pd/CuO/SiO₂, Rh/Ta₂O₅/Al₂O₃, Pd—Re/ZSM-5, Pt—W/Al₂O₃, Pt—Co/Mn₃(PO₄)₂, or any combination thereof.

39. The catalyst mixture of claim 27, wherein the catalyst mixture is Pt—W/Al₂O₃, and the Pt is present in the catalyst mixture at a concentration of about 5% by weight, and the W at a concentration of about 20% by weight.

40.-50. (canceled)
